(19) United States
(12) Patent Application Publication
(10) Pub. No.: US 2026/0102585 A1
(43) Pub. Date: Apr. 16, 2026

(54) LUMEN PLUG WITH ADHESIVE FLOW PATH AND RELATED SYSTEMS AND METHODS

(71) Applicant: Merit Medical Systems, Inc., South Jordan, UT (US)

(72) Inventor: Christopher Cindrich, Highland, UT (US)

(21) Appl. No.: 19/359,473

(22) Filed: Oct. 15, 2025

Related U.S. Application Data
(60) Provisional application No. 63/708,167, filed on Oct. 16, 2024.

(51) Int. Cl. A61M 25/00

(52) U.S. Cl. CPC A61M 25/0097

(57) ABSTRACT

A hub assembly is provided. The hub assembly including a main body including a cavity, an aperture extending from an exterior surface of the main body to the cavity, and an attachment component disposed within the cavity. The attachment component includes a vent fluidly connected to the aperture, and a distal portion configured to fixedly attach a catheter lumen. The flow path is fluidly connected to the aperture and defined by an exterior surface of the attachment component and an interior surface of the cavity.

### RELATED CASES

This application claims priority to United States Provisional Application No 63/708167 filed on October 16, 2024 and titled “LUMEN PLUG WITH ADHESIVE FLOW PATH AND RELATED SYSTEMS AND METHODS,” which is hereby incorporated by reference in its entirety.

### TECHNICAL FIELD

The present application relates to stent prostheses, guidewires, catheters and methods of using the same.

### BRIEF DESCRIPTION OF THE SEVERAL VIEWS OF THE DRAWINGS

The embodiments disclosed herein will become more fully apparent from the following description and appended claims, taken in conjunction with the accompanying drawings. The drawings depict only typical embodiments, which embodiments will be described with additional specificity and detail in connection with the drawings in which:

FIG. 1 illustrates a perspective view of a catheter system, according to embodiments herein.

FIG. 2 illustrates a perspective view of a hub assembly of the catheter system of FIG. 1, according to embodiments herein.

FIG. 3 illustrates a perspective internal view of the catheter system of FIG. 1, according to embodiments herein.

FIG. 4 illustrates a perspective internal view of the catheter system of FIG. 1, according to embodiments herein.

FIG. 5 illustrates a lateral view of an attachment component of the catheter system of FIG. 1, according to embodiments herein.

FIG. 6 illustrates a lateral view of the attachment component of FIG. 5, according to embodiments herein.

FIG. 7A illustrates a perspective cross-sectional view along line 7A-7A of FIG. 3 of the catheter system of FIG. 1, according to embodiments herein.

FIG. 7B illustrates a cross-sectional view along line 7B-7B of FIG. 3 of the catheter system of FIG. 1, according to embodiment herein.

FIG. 8 illustrates a lateral view of an attachment component of the catheter system of FIG. 1, according to embodiments herein.

FIG. 9 illustrates a lateral view of the attachment component of FIG. 8, according to embodiments herein.

FIG. 10A illustrates a perspective cross-sectional view along line 10A-10A of FIG. 3 of the catheter system of FIG. 1, according to embodiments herein.

FIG. 10B illustrates a cross-sectional view along line 10B-10B of FIG. 3 of the catheter system of FIG. 1, according to embodiment herein.

### DETAILED DESCRIPTION

Within medical device manufacturing, adhesives are often used to secure a configuration or assembly of multiple components. For instance, adhesives play a role in ensuring that a catheter sheath is securely affixed to a hub or handle assembly. Adhesives may also be used to secure subcomponents of a hub to each other and/or to other parts. By creating a reliable and durable bond between components that may otherwise be difficult to secure, adhesives portend stability and functionality to various catheter systems, structures, assemblies, as well as associated medical procedures and treatments.

Adhesive bonds within catheter assemblies must meet certain criteria to function properly within a medical setting. Internal adhesive bonds must be of sufficient strength to withstand the rigors of various medical procedures. These can include insertion, manipulation, removal, etc., of the device, without compromising overall performance or safety.

One factor influencing a bond's effectiveness within a medical device is the adhesive's distribution or flow during the application process. The manner in which an adhesive spreads and fills the interface between two components (e.g., a sheath and hub or handle assembly) significantly affects the strength and uniformity of the resulting bond. Proper distribution ensures that the adhesive adequately coats and bonds the mating surfaces, promoting strong adhesion and bond strength.

On one hand, if adhesive distribution is inadequate or uneven, weak spots or areas of insufficient bonding can occur, compromising the overall integrity of the bond. On the other hand, surplus or excessive flow can lead to potential issues such as overflow and penetration into areas where the adhesive should not reach. For example, overflowing adhesive can create excess material buildup (e.g., within a lumen of a catheter), which may interfere with the functionality of the device. For instance, if adhesive penetrates into internal channels, lumens, or other sensitive areas, surplus adhesive can obstruct fluid flow, erroneously transfix components, or otherwise interfere with a device's operation. This, in turn, can create performance issues or potential safety hazards.

Adhesive is typically applied via dispensing equipment. In one conventional approach, adhesive is dispensed onto the components, and then components are aligned and pressed together, and the adhesive is allowed to cure. Alternatively, the components may be aligned first, before the adhesive is dispensed. Once the components are properly aligned, adhesive can be dispensed onto the mating surfaces, and the assembly and curing proceed as usual.

Conventional methods for delivering adhesive to a medical device assembly possess some limitations and challenges. For instance, applying adhesives after assembly presents a particular issue related to space constraints and clearance limitations. In some cases, the available space within the assembly may be insufficient for adequate air flow, leading to an environment resembling an air trap, or airlock, that inhibits the ability of the adhesive to flow freely and uniformly distribute across the mating surfaces. As discussed, this restricted flow can result in uneven adhesive coverage and inadequate bonding, compromising the integrity and strength of the bond. Additionally, limited clearance between components may hinder access for adhesive application, further exacerbating the problem.

As discussed, once the components are assembled, the issue of over-penetration of the adhesive into unintended areas can be significant. Unmanaged distribution of adhesive can infiltrate non-targeted internal channels or lumens. Over-penetration poses a substantial risk to the device's performance, and exacerbates challenges associated with adhesive bonding in medical device manufacturing.

Aspects and implementations of the present disclosure address these and other challenges by providing methods and mechanism to facilitate, manage, and direct adhesive flow such that adhesive distribution and containment are enhanced. In some embodiments, an attachment, or alignment, component within a catheter hub assembly can include integrated flow paths for managing and containing the flow of an adhesive. An attachment component can further include one or more air vents to facilitate the flow and penetration of adhesive into the flow path, or channel, while containing adhesive to the targeted areas.

The phrase “coupled to” is broad enough to refer to any suitable coupling or other form of interaction between two or more entities, including mechanical, fluidic and thermal interaction. Thus, two components may be coupled to each other even though they are not in direct contact with each other. The phrase “fluid communication” is used in its ordinary sense, and is broad enough to refer to arrangements in which a fluid (e.g., a gas or a liquid) can flow from one element to another element when the elements are in fluid communication with each other.

The terms “proximal” and “distal” are opposite directional terms. As used herein, the distal end of a device or component is the end of the component that is furthest from the physician during ordinary use. The proximal end refers to the opposite end, or the end nearest the physician during ordinary use. For example, the proximal end of an introducer sheath used in minimally invasive vascular treatment is the end accessible to a practitioner during use, while the distal end is disposed within a patient’s vascular system when the sheath is placed into such a patient.

An assembler may be any person, system, or machine used in the manufacture of the introducer sheaths.

Embodiments may be understood by reference to the drawings, wherein like parts are designated by like numerals throughout. The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

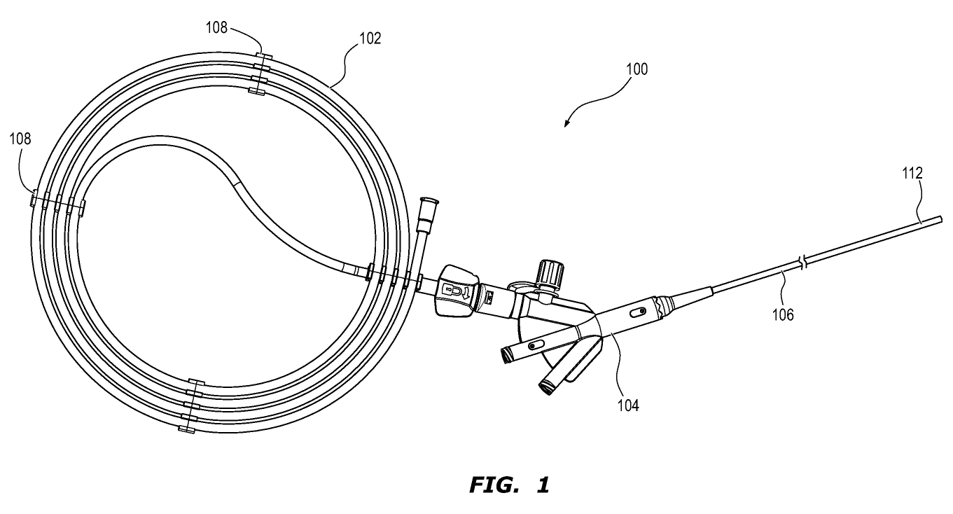

FIG. 1 illustrates a perspective view of a catheter system 100 according to embodiments herein In the illustrated embodiment the catheter system 100 includes a sheath 106 in fluid communication with a hub assembly 104 In certain embodiments a coiled member 102 may be fluidly connected to the sheath 106 via the hub assembly 104 In some embodiments the coiled member 102 can be a hollow shaft that is flexible and may be configured to retain a guidewire or other member within the hollow shaft of the coiled member 102 The coiled member 102 can be coiled and held together via clips 108 The coiled member 102 can be fluidly connected to a proximal side of the hub assembly 104 The sheath 106 can be fluidly connected to a distal side of the hub assembly 104 In some embodiments the sheath 106 can extend distally from a distal end of the hub assembly 104 to a distal end 112 or delivery end of the catheter system 100 The hub assembly 104 can provide a proximal user input with one or more components configured to allow a practitioner to deploy or otherwise manipulate further devices eg a prosthesis guidewire etc disposed within the catheter system 100.

During use, the hub assembly 104 can be disposed outside of a patient’s body, while the sheath 106 can be advanced to a treatment location within the patient’s body. For example, the sheath 106 may be advanced from an insertion site (such as, for example, a femoral or jugular insertion site) to a treatment location within the vasculature. The catheter system 100 and/or the sheath 106 can be configured to be advanced through bends, turns, or other structures within the anatomy of the vasculature.

In some embodiments, the sheath 106 can include a braided wire frame and an impermeable material. In some cases, the braided wire frame can be of varying levels of rigidity. For example, a distal portion of the wire frame can have a greater picks/inch braid and/or rigidity than a proximal portion of the wire frame.

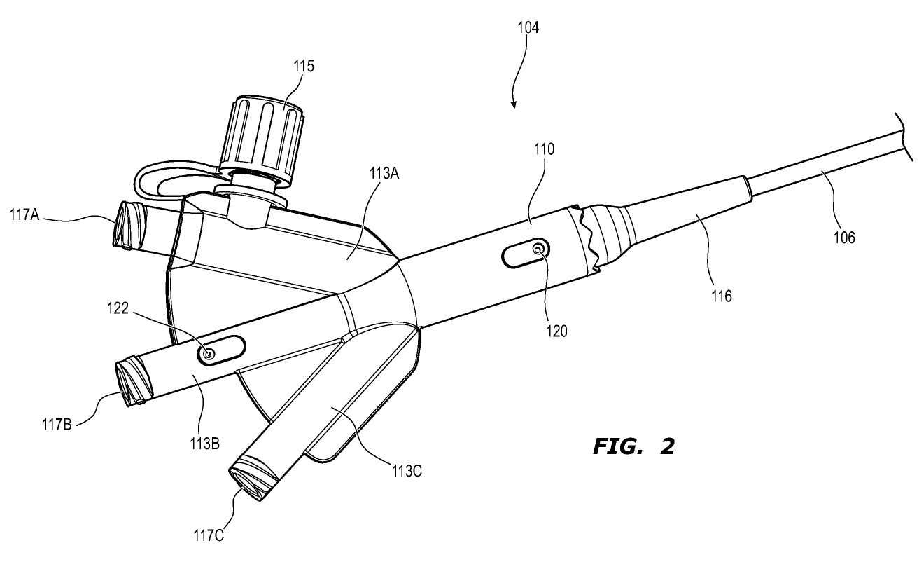

FIG. 2 illustrates a perspective view of the hub assembly 104 of the catheter system 100 of FIG. 1 according to embodiments herein In some embodiments the hub assembly 104 can include an internal cavity defined by cavities corresponding to one or more fluid pathways eg fluid pathways 113A 113B 113C andor a cavity of a main body 110 of the hub assembly 104 For example the fluid pathways 113A 113B 113C and corresponding cavities can extend from a distal end 116 of the hub assembly 104 through a cavity of the main body 110 to a proximal end of the hub assembly 104 In some embodiments the fluid pathways 113A 113B 113C and corresponding cavities can fluidly connect eg transfer fluid from a proximal connection end eg connection portions 117A 117B 117C to distal end 116.

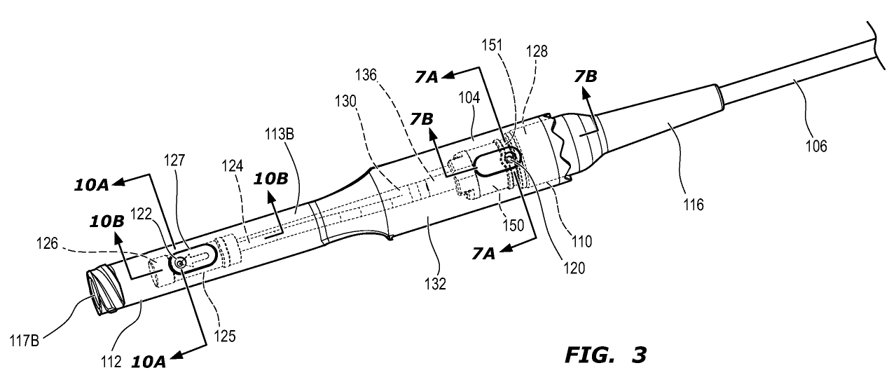

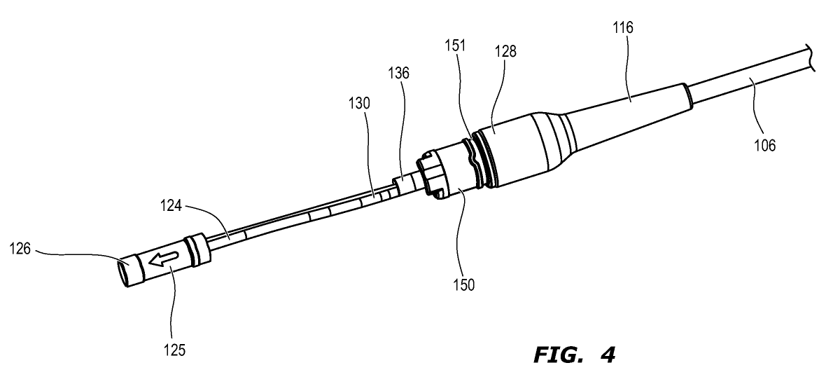

In some embodiments, the cavities of the fluid pathways 113A–113C can connect to a cavity of the main body 110 and be used to fluidly connect to corresponding, distinct, lumens (not seen in FIG. 2) within the sheath 106. In some cases, distinct lumens of the sheath 106 can include transition members (discussed with respect to FIGS. 3-4) and attachment components (seen in FIGS. 3-4) for connecting one or more lumens of the sheath 106 to a specific fluid pathway. Thus, the fluid pathways 113A–113C, corresponding connection portions, transition members, attachment components, and lumens within the sheath 106 can be fluidly connected and be used to transfer fluids from a proximal end to a distal end of the catheter system 100 (or vice-versa).

Similarly, in some embodiments, one or more elongate members (e.g., guidewires, inflation lumens, etc.) can be passed through catheter system 100 from a proximal end to a distal end (or vice-versa). In some embodiments, any number of fluid pathways, corresponding cavities, connection portions, transition members, attachment components, and lumens can be included in the hub assembly 104.

In some embodiments, hub assembly 104 can further include a side port 115 fluidly connected to a fluid pathway of the hub assembly 104. Side port 115 can include a cap for closing port and/or may include one or more valves. In some embodiments the side port 115 may be used to flush one or more portions of the hub assembly 104.

During manufacturing and assembly, transition members (as will be discussed with respect to FIGS. 3-4) and attachment components can be used to fluidly connect lumens of the sheath 106. For instance, the hub assembly 104 can be assembled and fixed into place such that fluid connections extend from connection portions 117A–117C to the distal end of the sheath 106. As mentioned, during manufacturing, internal components of the hub assembly 104 can be arranged and secured via adhesives.

In some cases, after assembly or placement of the hub assembly 104, adhesive can be dispensed through apertures (e.g., apertures 120, 122) in an outer wall of the hub assembly 104. In some embodiments, apertures 120 and 122 can be used to introduce adhesive into the interior cavity of a fluid pathway of the hub assembly 104. Through apertures 120 and 122, adhesive can be directed internally within the hub assembly 104 to flow and bond attachment components, transition members, lumens, structures, etc., to hub assembly 104. Thus, in some embodiments, the hub assembly 104 can be hollow, and include apertures selectively located on an exterior surface of the hub assembly 104 to facilitate attachment of internal components at that location. In some embodiments, the hub assembly 104 can include any number of apertures in any configuration or location, to introduce adhesive as is feasible or appropriate, internally to the hub assembly 104.

FIGS. 3 and 4 illustrate perspective internal views of the catheter system of FIG. 1 according to embodiments herein and will be described jointly FIG. 3 illustrates a perspective internal view of the catheter system of FIG. 1 according to embodiments herein FIG. 4 illustrates a perspective internal view of the catheter system of FIG. 1 according to embodiments herein The internal components of FIG. 4 are illustrated in broken lines in FIG. 3

FIGS. 3 and 4 illustrate only one fluid pathway (e.g., fluid pathway 113B) and corresponding cavity (e.g., cavity 127). One of ordinary skill in the art, having the benefit of this disclosure, will note that the techniques and embodiments described with respect to FIGS. 3–4 can be applied to additional fluid paths and connections (e.g., transition members, attachment components, sheaths, etc.). E.g., the hub assembly 104 as seen and described can include one, or more, or any number of fluid pathways, cavities, lumens, and distinct fluid connections as is feasible or appropriate. Each distinct fluid connection can be fluidly connected through the hub assembly 104 to a distal end of the catheter sheath 106.

Within the illustrated embodiment of FIGS. 3-4, transition members 124, 130 can be fluidly connected to respective fluid pathway(s) of the hub assembly 104. For instance, the transition member 124 can be connected to the fluid pathway 113B via an attachment component 126. In some embodiments, the attachment component 126 can serve to center, align, and/or fix the transition member 124 within the cavity 127 of the fluid pathway 113B. For instance, the transition member 124 can connect to an axially central location of the attachment component 126, and the attachment component 126 can be centered within the internal cavity 127 of fluid pathway 113B. Otherwise stated, longitudinal axes of the attachment component 126 and the fluid pathway 113B can align. A fluid connection formed between the cavity 127 of the fluid pathway 113B and the transition member 124 can facilitate fluid transfer to a lumen of the sheath 106 and to the distal end 112 of the catheter system 100.

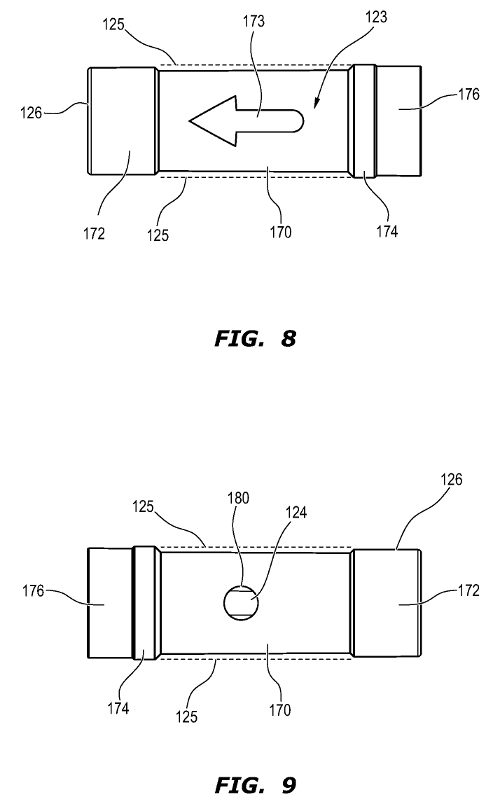

To facilitate adhesive bonding, the attachment component 126 can isolate or define a partition 125 (e.g., cavity, space, void, or channel) of the cavity 127. The partition 125 can then be targeted for adhesive flow and bonding. A radially interior limit of the partition 125 can be defined by the attachment component 126, and a radially exterior limit can be defined by an outer wall of the cavity 127, or the fluid pathway 113B. In some embodiments, distal and proximal, longitudinal ends to the partition 125 can be defined by the attachment component 126 (as will be further discussed with respect to FIGS. 8-10B). In some embodiments, the partition 125 can extend all or a portion of the circumference of the attachment component 126 and cavity 127.

The aperture 122 can be used to delivery an adhesive to the partition 125. In some embodiments, the aperture 122 can extend through from an external surface of the hub assembly 104, to the internal cavity 127, and the partition 125. Upon flowing into the partition 125, adhesive can set and bond the attachment component 126 and the transition member 124 to the cavity 127 and the fluid pathway 113B. In such a way, adhesive can be administered to the hub assembly 104 (e.g., to partition 125) in a targeted and controlled manner.

Similar to the attachment component 126, the partition 125, and the aperture 122, a second attachment component and aperture can be used to center, align, and/or fix sheath 106 to the hub assembly. For instance, in some embodiments, an attachment component 150 and an aperture 120 can be employed to transfix the sheath 106 to an interior cavity 132 of the main body 110. For instance, the sheath 106 can connect to an axially central location of the attachment component 150, and the attachment component 150 can be centered within the internal cavity 132 of the main body 110. Otherwise stated, longitudinal axes of the attachment component 150 and the cavity 132 can align. A fluid connection formed between the cavity 132, lumens within the sheath 106, and/or transition members can facilitate fluid transfer to a lumen of the sheath 106 and to the distal end 112 of the catheter system 100.

As previously discussed, the attachment component 150 can isolate or define a partition 151 of the cavity 132 of the main body 110. The partition 151 can be targeted for adhesive flow and bonding through the aperture 120. A radially interior limit of the partition 151 can be defined by the attachment component 150, and a radially exterior limit can be defined by an outer wall of the cavity 132, or inner wall of the main body 110. In some embodiments, distal and proximal, longitudinal ends to the partition 151 can be defined by the attachment component 150 (as will be further discussed with respect to FIGS. 5-7B. In some embodiments, the partition 151 can extend all or a portion of the circumference of the attachment component 150 and the cavity 132.

The aperture 120 can be used to delivery an adhesive to the partition 151. In some embodiments, the aperture 120 can extend through from an external surface of the hub assembly 104, to the internal cavity 132, and the partition 151. Upon flowing into the partition 151, adhesive can set and bond the attachment component 150 to the cavity 132 and the main body 110. In such a way, adhesive can be administered to the hub assembly 104 (e.g., to partition 151) in a targeted and controlled manner.

In some embodiments, the partition (e.g., partition 125 and/or 141) formed by an attachment component can include a specific, or controlled flow path. For instance, an attachment component can include a channel on a radially exterior surface of the attachment component. The channel can define a flow path that is part of the corresponding partition. In such a way, further control and management of the flow of adhesive can be performed.

FIG. 5 illustrates a lateral view of an attachment component 150 of the catheter system 100 of FIG. 1 according to embodiments herein In the illustrated embodiment the attachment component 150 includes a channel 144 for defining the partition 151 for the adhesive A portion of channel 144 can be defined by ridges 142 and 146 on an exterior surface 148 of the attachment component 150 The ridges 142 and 146 can extend radially from the exterior surface 148 of the attachment component 150.

As described, the channel 144 can define the partition 151 together with an internal wall of a cavity in which the attachment component 150 is placed. In the illustrated embodiment, the channel 144 can define a flow path, for receiving and containing adhesive as it disperses throughout the partition 151.

The attachment component 150 can include a bend 147 that can align with the aperture 120 (not seen in FIG. 5) of the hub assembly 104. An introduction point 121 may align with the aperture 120 and may service for an introduction point for the adhesive into the channel 144. The bend 147 enlarges the area of the introduction point 121 to receive the adhesive through the aperture 120. In cases, the adhesive delivered through the aperture 120 aligned with the introduction point 121 can distribute evenly throughout channel 144. For instance, the adhesive can distribute from the introduction point 121 in both lateral directions (e.g., relative to a longitudinal axis of attachment component 150) as illustrated by arrows A1.

As discussed, in some embodiments the adhesive flow path and the channel 144 can extend a portion, substantially, or all, of the circumference of the attachment component 150. As will be discussed further with respect to FIG. 6, the adhesive can first fill the flow path defined by the channel 144 and the hub assembly 104, and secondly flow into the overflow area (e.g., an overflow area 140).

FIG. 6 illustrates a lateral view of the attachment component 150 of FIG. 5 according to embodiments herein In the illustrated embodiment an opposite side of the attachment component 150 (when compared to FIG. 5) is shown.

As illustrated in the embodiment of FIG. 6, the ridge 146 can extend radially around an entire circumference of the attachment component 150. The ridge 146 can define a distal end of the channel 144 and the adhesive flow path. The ridge 142 can define a proximal end of the channel 144, and a proximal end for that portion of the adhesive flow path.

As seen, the ridge 142 can include a pair of breaks 156, 158. The pair of breaks 156, 158 can be placed substantially opposite the bend 147 (seen in FIG. 5). In other words the pair of breaks 156, 158 may be diametrically opposed the aperture 120. The pair of breaks 156, 158 can fluidly connect the channel 144 to the overflow area 140. For instance, on introduction of adhesive from the aperture 120 of the hub assembly 104, adhesive can flow into the channel 144, and distribute equally in both lateral directions such that adhesive distributes equally throughout channel 144.

In cases where adhesive has flowed in equal directions into channel 144, adhesive can flow into the pair of breaks 156, 158 as illustrated by arrows A2. The adhesive can be redirected from a lateral direction to a longitudinal direction into the overflow area 140 in equal (or substantially equal) portions from the pair of breaks, 156, 158. A proximal ridge 149 or raised portion can serve as a proximal end, or bound, on the partition 151 targeted for delivery of adhesive to limit longitudinal flow of adhesive. The adhesive may then flow as illustrated by arrows A3 toward each other and away from each other. The adhesive may then flow laterally toward each other as illustrated by arrows A4 in FIG. 5.

Thus, as adhesive equilibrates and cures, the attachment component may be bound, or a bond may be formed substantially, and/or entirely, around the circumference of the attachment component 150. Such a bond may enhance sealing or binding of the attachment component to the hub assembly 104. Thus, the attachment component 150 may facilitate bonding or binding an elongate member or sheath to the hub assembly, while enhancing control and managing the spread and penetration of adhesive.

To facilitate flow of the adhesive throughout channel 144 and overflow area 140, a vent 160 may rest on and through the proximal ridge 149 of the attachment component 150. The vent 160 may be an aperture leading to an internal lumen or bore of the attachment component 150. As shown, the vent 160 may allow air to escape from the volume targeted for adhesive. E.g., air within the channel 144 and/or the overflow area 140 may escape through the vent 160, as adhesive is being introduced. This can prevent any air bubbles or pockets from forming, and allows for smooth and equal distribution of adhesive. In some cases, the vent 160 can be raised, or offset from exterior surface 148 such that adhesive does not pass into the inner bore of the attachment component 150, or into any other components of the assembled hub assembly 104.

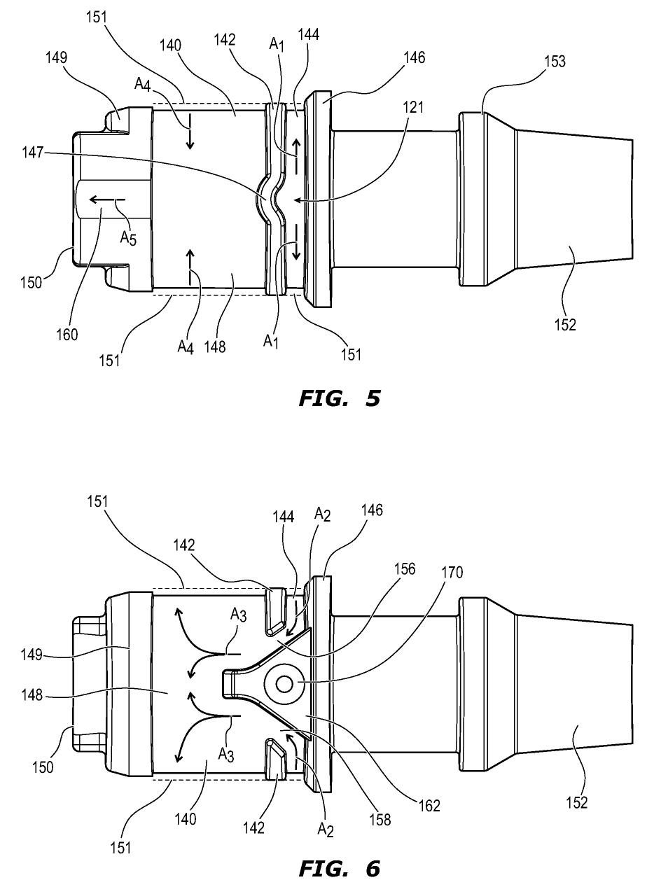

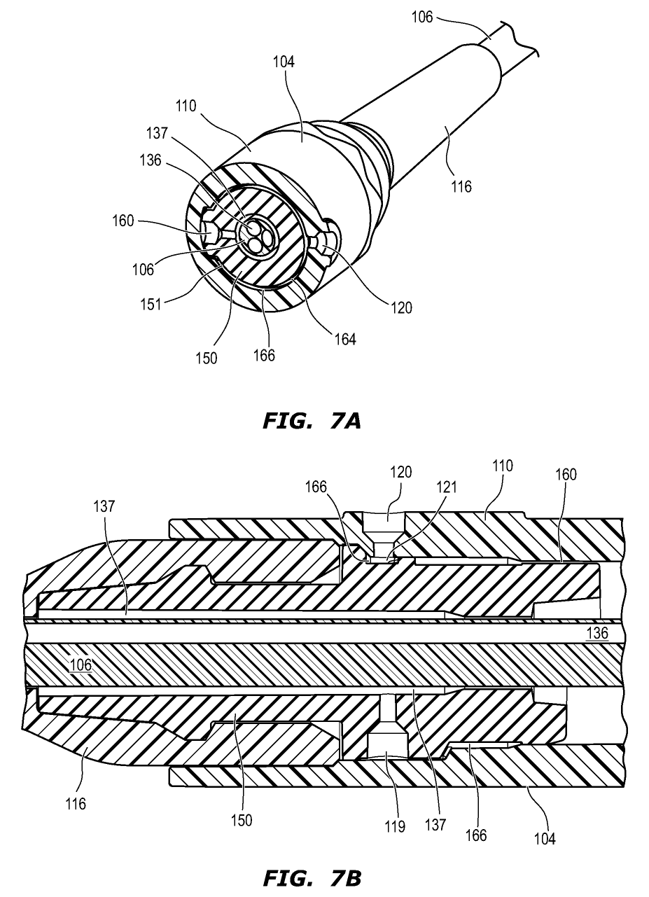

FIG. 7A illustrates a perspective cross-sectional view along line 7A-7A of FIG. 3 of the catheter system 100 of FIG. 1 according to embodiments herein As seen in illustrated embodiment the attachment component 150 can rest within the main body 110 of the hub assembly 104 The sheath 106 or a portion of the sheath 106 including lumens 136 can pass through the distal end 116 and attach to the attachment component 150.

The partition 151 can be a void or cavity formed or defined by the attachment component 150 and the main body 110. In some embodiments, an inner wall 166 or features, of the attachment component 150 can form a radially interior limit of the partition 151, and an outer (inner according to some perspectives) wall 164 of the main body 110 can form a radially exterior limit to the partition 151.

FIG. 7A illustrates a perspective cross-sectional view along line 7A-7A of FIG. 3 of the catheter system 100 of FIG. 1, according to embodiments herein. A flow path can be formed, extending from the aperture 120 of the main body 110 into the partition 151. As previously discussed, the partition 151 can extend (e.g., through the channel 144) around the circumference of the attachment component 150. The adhesive can be delivered through the aperture 120 into the flow path and/or the partition 151. The vent 160 can allow air to escape into an inner lumen 137 of the attachment component 150, as adhesive traverses the channel 144. As illustrated in FIG. 7B, the size of the vent 160 is design to allow air to escape but not to allow the adhesive to advance far into the vent 160 due to viscosity of the adhesive. In some embodiments, the rate or amount of adhesive may be controlled to ensure that too much adhesive is not introduced into the flow path.

An aperture 119 may be used to couple the attachment component 150 to the sheath 106. The aperture 119 may disposed on a raised portion 162 of the attachment component 150. Adhesive may be introduced into the aperture 119 and the adhesive may flow around the circumference of the sheath 106 and the adhesive may cure adhering the attachment component 150 to the sheath 106. The adhesive flow path from aperture 119 is different and separate from the adhesive flow path of aperture 120.

FIG. 8 illustrates a lateral view of the attachment component 126 of the catheter system of FIG. 1 according to embodiments herein As seen in the illustrated embodiment the attachment component 126 can include a proximal raised portion 172 and a distal raised portion 174 that engage with the wall 164 of the cavity As discussed the attachment component 126 can be configured to be centered within a fluid pathway or cavity of the hub assembly 104.

As seen, the attachment component 126 can define the partition 125 of a void or cavity surrounding the attachment component 126. A circumferential surface 170 can define an interior limit to the partition 125. The raised portion 172, 174 can define proximal and distal limits, respectively. Indicia 173 on the attachment component 126 can aid when an assembler is forming the assembly.

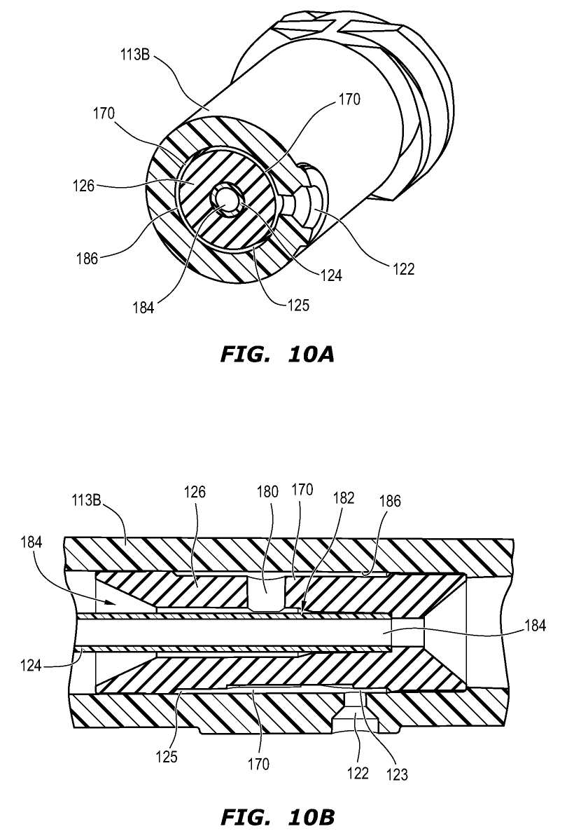

As will be further discussed with respect to FIGS. 9-10B, adhesive may enter the aperture 122 of the hub assembly 104 directly into partition 125 of the attachment component 126 at an introduction point 123. The partition 125 can rest between raised portions 172, 174, which can serve to contain the adhesive introduced through the aperture 122. The partition 125 can extend around a circumference of the attachment component 126, and serve to retain adhesive within a controlled space. Thus, the attachment component 126 can facilitate bonding or binding an elongate member or lumen to the hub assembly 104, while controlling or managing the spread and penetration of the adhesive.

FIG. 9 illustrates a lateral view of the attachment component 126 of FIG. 8 according to embodiments herein In the illustrated embodiment an opposite side of the attachment component 126 eg an opposite view when compared to FIG. 8is shown As illustrated in the embodiment of FIG. 9 raised portions 172 can extend radially around a portion or an entire circumference of the attachment component 126 Thus the partition 125 can extend radially around a portion or an entire circumference of the attachment component 126.

On introduction of the adhesive from the aperture 122 of the hub assembly 104 onto the introduction point 123, adhesive can flow into the partition 125 and distribute equally in all directions (e.g., clockwise and counterclockwise, and longitudinally along the attachment component). Thus, the adhesive may later bind, and form a bond substantially, and/or entirely, around the circumference of the attachment component 126. Such a bond may more strongly seal the attachment component to the hub assembly. Thus, attachment component 126 can facilitate bonding or binding an elongate member or lumen to the hub assembly 104, while controlling or managing the spread and penetration of adhesive.

To facilitate flow of the adhesive to partition 125, once the adhesive reaches aperture 180, the adhesive enters the aperture 180 and flows circumferentially around the transition member 124 to attached the transition member 124 to the attachment component 126.

FIG. 10A illustrates a perspective cross-sectional view of the attachment component 126 taken along line 10A-10A of FIG. 3 of the catheter system of FIG. 1 according to embodiments herein As seen in the illustrated embodiment the attachment component 126 can rest within a lumen of the fluid pathway 113B of the hub assembly 104.

The partition 125 can be a void or cavity formed or defined by the attachment component 126 and the fluid pathway 113B. In some embodiments, the circumferential surface 170 or features, of the attachment component 126 can form a radially interior limit of the partition 125, and an outer (inner according to some perspectives) wall 186 of the fluid pathway 113B can form a radially exterior limit to partition 125.

A flow path for adhesive can be formed, extending from aperture 122 of fluid pathway 113B into partition 125. As previously discussed, partition 125 can extend around the circumference of the attachment component 126. Adhesive can be delivered through aperture 122 into the flow path and/or partition 125.

FIG. 10B illustrates a perspective cross-sectional view of the attachment component 126 taken along line 10A-10A of FIG. 3 of the catheter system 100 of FIG. 1, according to embodiments herein. As shown, adhesive enters the aperture 122 to introduction point 123 and the adhesive flows circumferentially around the attachment component 126 and into aperture 180. Once the adhesive enters aperture 180, the adhesive flows circumferentially around the transition member 124. The adhesive is bound by a tapered end 182 and any air escapes through vent 184 as the adhesive is delivered.

Devices, including hub assemblies, catheters, and related components as described above are all within the scope of this disclosure. Additionally, any methods for assembling, forming, and/or bonding the components as described above are likewise within the scope of the present disclosure.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” or “embodiments” means that a particular feature, structure, or characteristic described in connection with that embodiment is included in at least one embodiment. Thus, the quoted phrases, or variations thereof, as recited throughout this specification are not necessarily all referring to the same embodiment.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A hub assembly, comprising:
a main body comprising a cavity;
a first aperture extending from an exterior surface of the main body to the cavity;
a first attachment component disposed within the cavity, the first attachment component comprising:
a first vent fluidly connected to the first aperture; and
a first flow path fluidly connected to the first aperture and defined by an exterior surface of the first attachment component and an interior surface of the cavity.

2. The hub assembly of claim 1, wherein the exterior surface of the first attachment component comprises a channel extending circumferentially along the exterior surface of the first attachment component.

3. The hub assembly of claim 1, wherein the first flow path extends around an inner circumference of the cavity.

4. The hub assembly of claim 1, wherein the first flow path comprises a pair of breaks that are diametrically opposed the first aperture and the pair of breaks redirect the first flow path from a lateral flow to a longitudinal flow.

5. The hub assembly of claim 1, wherein the vent is disposed between the hub assembly and the first attachment component that allows air to escape from the first flow path.

6. The hub assembly of claim 5, wherein the vent longitudinally aligns with the first aperture.

7. The hub assembly of claim 1, wherein the first attachment component further comprises an overflow area fluidly connected to the first flow path via a pair of breaks in the flow path.

8. The hub assembly of claim 7, wherein the overflow area comprises a proximal ridge configured to limit the longitudinal flow.

9. The hub assembly of claim 1, further comprising:
a second aperture extending from the exterior surface of the main body to the cavity;
a second attachment component disposed within the cavity, the second attachment component comprising:
a body with a distal portion and a proximal portion that engage with the interior surface of the cavity and a circumferential surface disposed between the distal portion and the proximal portion radially offset from the interior surface of the cavity; and
a third aperture defined by the circumferential surface that leads to a cavity of the second attachment component;
a second flow path fluidly connected to the second aperture and defined by the circumferential surface of the second attachment component and the interior surface of the cavity and enters the third aperture.

10. The hub assembly of claim 9, wherein the third aperture is longitudinally offset from the second aperture.

11. A system, comprising:
a catheter hub body comprising a cavity;
an aperture extending from an exterior surface of the catheter hub body to the cavity;
an attachment component disposed within the cavity, the attachment component comprising:
a vent fluidly connected to the aperture; and
a flow path fluidly connected to the aperture and defined by an exterior surface of the attachment component and an interior surface of the cavity.

12. The system of claim 11, wherein the exterior surface of the attachment component comprises a channel extending circumferentially along the exterior surface of the attachment component.

13. The system of claim 11, wherein the flow path extends around an inner circumference of the cavity.

14. The system of claim 11, wherein the flow path comprises a pair of breaks that are diametrically opposed the aperture and the pair of breaks redirect the flow path from a lateral flow to a longitudinal flow.

15. The system of claim 11, wherein the vent is disposed between the attachment component and the aperture.

16. The system of claim 11, wherein the vent longitudinally aligns with the aperture.

17. The system of claim 11, wherein the attachment component further comprises an overflow area fluidly connected to the flow path via a pair of breaks in the flow path.

18. A method of manufacturing a medical device, comprising:
forming an assembly comprising an attachment component disposed within a cavity of a catheter hub body;
introducing an adhesive through an aperture of the catheter hub body to a flow path defined by an exterior surface of the attachment component and an interior surface of the cavity, such that adhesive traverses the flow path in both a clockwise direction and a counterclockwise direction; and
allowing the adhesive to cure.

19. The method of claim 18, wherein introducing the adhesive through the aperture of the catheter hub body comprises introducing adhesive at a rate or amount such that the adhesive traverses an entirety of the flow path.

20. The method of claim 18, wherein introducing the adhesive through the aperture of the catheter hub body comprises introducing adhesive at a rate or amount such that the adhesive enters an overflow area defined by the exterior surface of the attachment component and the interior surface of the cavity.
